(19) United States
(12) Patent Application Publication
(10) Pub. No.: US 2002/0022594 A1
(43) Pub. Date: Feb. 21, 2002

(54) Bax fragment induced tumor cell death

(76) Inventors: Ping Dou, Tampa, FL (US); Gui Gao, Tampa, FL (US)

Correspondence Address: Kohn & Associates, Suite 410, 30500 Northwestern Highway, Farmington Hills, MI, 48334, US

(21) Appl. No.: 09/903,819

(22) Filed: Jul. 11, 2001

Related U.S. Application Data
(60) Provisional application No. 60/217,264, filed on Jul. 11, 2000.

(51) Int. Cl. A61K 38/17

(52) U.S. Cl. 514/12

(57) ABSTRACT

The present invention relates treatment of tumors using a Bax protein fragment to induce apoptosis. More specifically, the present invention relates to the use of the Bax amino terminus 18 kDa fragment to induce cytochrome c release and apoptosis in cancer cells. Further provided is the method of inducing cell death even in cancer cells overexpressing Bcl-2 oncoprotein. Further provided is a method using Bax/p18 of triggering cytochrome c release, activation of caspase-3, cleavage of poly(ADP-ribose) polymerase, fragmentation of DNA and induction of cell death. Further provided is a method of introducing Bax/p18 protein into a cancer cell via introducing Bax/p18 cDNA, to cause release of cytochrome c and induction of caspase-3 mediated apoptosis that is not blocked by overexpression of Bcl-2.

### CROSS-REFERENCE TO RELATED APPLICATIONS

This applications claims the benefit of priority under 35 U.S.C. Section 119(e) of United States Provisional Patent Application No. 60/217,264, filed Jul. 11, 2000, which is incorporated herein by reference.

### GRANT INFORMATION

National Institutes of Health, National Institute of Aging. Grant No. 5 R29 AG13300-06.

### BACKGROUND OF THE INVENTION

1. Field of the Invention

The present invention relates to treatment of tumors using a Bax protein fragment to induce apoptosis. More specifically, the present invention relates to the use of the Bax amino terminus 18 kDa fragment to induce cytochrome c release and apoptosis in cancer cells.

2. Description of Related Art

Homeostasis of cell number is achieved by balancing the processes of cell proliferation and cell death. Recent evidence suggests that dysregulation of cell cycle progression is probably one of the important events for the initiation of apoptosis (Song and Steller 1999). Apoptosis, an evolutionarily conserved form of cell suicide, is the process by which a cell will actively commit suicide under tightly controlled circumstances (Steller, H. Mechanisms and genes of cellular suicide. Science 1995; 267: 1445-1449.). Morphologically, apoptosis is characterized by shrinkage of the cell, dramatic reorganization of the nucleus, active membrane blebbing, and ultimate fragmentation of the cell into membrane-enclosed vesicles (apoptotic bodies) (Wylie, A. H., Kerr, J. F. R., Currie, A. R. Cell Death: The Significance of Apoptosis. Int. Rev. Cytol. 1980; 68: 251-306; Earnshaw, W. C. Nuclear Changes in Apoptosis. Curr. Opin. Cell Biol. 1995; 7: 337-343; Steller, H. Mechanisms and genes of cellular suicide. Science 1995; 267: 1445-1449).

Apoptosis occurs in three physiological stages, initiation, commitment and execution (Reed et al 1997). It has been proposed that the tumor suppressor p53 (8, 9) and the apoptosis regulator Bcl-2 family proteins (10-13) are involved in the apoptotic commitment in mammalian cells. Apoptotic execution in mammalian cells is initiated by specific caspase proteases, often followed by activation of endonucleases and consequent internucleosomal fragmentation of DNA (180-bp ladders) (5-7,14).

Mitochondria play an essential role in apoptotic commitment (Green, D. R. and Reed, J. C. Mitochondria and Apoptosis. Science 1998; 281: 1309-1312; Cory, S. and Adams, J. M. The Bcl-2 Protein Family: Arbiters of Cell Survival. Science 1998; 281: 1322-1326; Wang, H-. G. and Reed, J. C. Mechanisms of Bcl-2 Protein Function. Histol Histopathol 1998; 13: 521-530). Upon apoptosis stimulation, several key events occur in mitochondria, including the release of caspase activators (such as cytochrome c, procaspase-3 and apoptosis-inducing factor), disruption of electron transport, alteration of cellular reduction-oxidation potential, loss of mitochondrial transmembrane potential, and participation of pro- and antiapoptotic Bcl-2 family proteins (Green and Reed, 1998, Adams and Cory 1998, Gross et al 1999). Since the first discovery of the bcl-2 oncogene in 1985 (Tsujimoto, Y., Cossman, J., Jaffe, E., Croce, C. Involvement of the bcl-2 proto-oncogene expression of cellular sensitivity to tumor necrosis factor-mediated cytotoxicity. Oncogene 1985; 8: 1440-1443), at least 15 cellular homologs of Bcl-2 protein have been reported, including the proapoptotic proteins Bax, Bcl-XS, Bad, Bak, Bik, Bid and Hrk and the antiapoptotic proteins Bcl-XL, Mcl-1, A1/Bfl-1, Bcl-W, Nr-13, and Ced-9 (Green and Reed, 1998, Adams and Cory 1998, Gross et al 1999). Several Bcl-2 family proteins are located in the outer mitochondrial membrane, where they control release of cytochrome c into the cytosol. The cytochrome c release can be induced by expression of a proapoptotic member of Bcl-2 family (such as Bax, Bid), and inhibited by expression of an antiapoptotic Bcl-2 family member (such as Bcl-2, Bcl-XL) (Green and Reed, 1998, Adams and Cory 1998, Gross et al 1999). The ratio of pro- to antiapoptotic Bcl-2-like proteins (i.e., Bax/Bcl-2), therefore, determines whether a cell is committed to apoptotic death or not.

In the most current view, once cytochrome c is released from mitochondria, this commits the cell to die by either a rapid apoptotic mechanism or a slower necrotic process due to collapse of electron transport (Green and Reed 1998). The cytochrome c-induced apoptotic process involves Apaf-1-mediated caspase activation. The cytosolic cytochrome c interacts with Apaf-1, which induces association of Apaf-1 to procaspase-9, thereby triggering processing and consequent activation of caspase-9. The activated caspase-9 in turn cleaves a downstream effector caspase (such as caspase-3), initiating apoptotic execution (Green and Reed 1998; Thornberry, N. A. and Lazebnik, Y. Caspases: Enemies Within. Science 1998, 281: 1312-1316; Martin S J, Green D R. Protease activation during apoptosis: death by a thousand cuts? Cell 1995; 82: 349-352). At least 13 caspases have been identified and cloned (Thornberry, and Lazebnik 1998). Activation of effector caspases leads to apoptosis probably through the proteolytic cleavage of important cellular proteins (Earnshaw, W. C. Nuclear Changes in Apoptosis. Curr. Opin. Cell Biol. 1995; 7: 337-343; Steller, H. Mechanisms and genes of cellular suicide. Science 1995; 267: 1445-1449; Thornberry and Lazebnik 1998; Martin and Green 1995), such PARP (Lazebnik, Y. A, Kaufmann, S. H., Desnoyers, S., Poirier, G. G, Earnshaw, W. C. Cleavage of poly(ADP-ribose) polymerase by a proteinase with properties like ICE. Nature 1994; 371: 346-347), actin (Kayalar, C., Ord, T., Testa, M. P, Zhong, L. -T, Bredesen, D. E. Cleavage of actin by interleukin-1b-converting enzyme to reverse DNase I inhibition. Proc. Natl. Acad. Sci. USA 1996; 93: 2234-2238), sterol regulatory binding proteins (Wang, X., Zelenski, N. G., Yang, J., Sakai. J., Brown, M. S., Goldstein, J. L. Cleavage of sterol regulatory element binding proteins (SREBPs) by CPP32 during apoptosis. EMBO J. 1996; 15: 1012-1020) and DNA-dependent protein kinase (Song, Q., Lees-Miller, S. P., Kumar, S., Zhang, N., Chan, D. W., Smith, G. C. M., Jackson, S. P, Alnemri, E. S., Litwack, G., Khanna, K. K., Lavin M F. DNA-dependent protein kinase catalytic subunit: a target for an ICE-like protease in apoptosis. EMBO J. 1996; 15: 3238-3246). During apoptosis, the retinoblastoma protein (RB) is cleaved by a caspase activity into two major fragments, p68 and p48 (An, B., Dou, Q. P. Cleavage of retinoblastoma protein during apoptosis: an interleukin 1b-converting enzyme-like protease as candidate. Cancer Res. 1996; 56: 438-442; Fattman, C. L., An, B., Dou, Q. P. Characterization of interior cleavage of retinoblastoma protein in apoptosis. J. Cell. Biochem. 1997; 67: 399-408; Dou, Q. P, An, B., Antoku, K., Johnson, D. E. Fas stimulation induces RB dephosphorylation and proteolysis that is blocked by inhibitors of the ICE-like protease family. J. Cell. Biochem. 1997; 64: 586-594). Subsequently, other groups reported that during apoptosis RB was also cleaved from its C-terminus by a caspase-3-like protease (Janicke, R. U., Walker, P. A., Lin, X. Y., Porter A G. Specific cleavage of the retinoblastoma protein by an ICE-like protease in apoptosis. EMBO J. 1996; 15: 6969-6978; Tan X, Martin S J, Green D R, Wang Y J. Degradation of retinoblastoma protein in tumor necrosis factor and CD95-induced cell death. J. Biol. Chem. 1997; 272: 9613-9616). Previously, a late Bax cleavage activity, which was detected several hours after DNA fragmentation, was reported to be a calpain-like, but not caspase-like, activity (Wood and Newcomb, 1999; Wood et al., 1998). The Bax cleavage site for the late calpain activity was identified to be around amino acids ³⁰FIQD³³ of Bax (Wood, et al., 1998).

Regulation of apoptosis is deranged in most, if not all, human cancers (Fisher D E. Apoptosis in cancer therapy: crossing the threshold. Cell 1994; 78: 539-542). Many human cancers are resistant to induction of apoptosis (Fisher 1994; Harrison, D. J. Molecular mechanisms of drug resistance in tumors. J. Patho. 1995; 175: 7-12; Milner, J. DNA damage, p53 and cancer therapies. Nature Med. 1995; 1: 789-880) at least partially due to inactivation of the tumor suppressor protein p53 (Milner 1995) or overexpression of the Bcl-2 (Reed J. C. Bcl-2 and the Regulation of Programmed Cell Death. J. Cell Biol. 1994; 124: 1-6) or Bcr-Abl oncoprotein (Bedi, A., Zehnbauer, B. A., Barber, J. P., Sharkis, S. J., Jones, R. J. Inhibition of apoptosis by Bcl-ABL in chronic myeloid leukemia. Blood 1994; 83: 2038-2044). Indeed, higher Bcl-2/Bax ratio correlates with poor therapeutic responsiveness to radio or chemotherapy in patients with prostate (Mackey, T. J., Borkowski, A., Amin, P., Jacobs, S. C., Kyprianou, N. Bcl-2/bax ratio as a predictive marker for therapeutic response to radiotherapy in patients with prostate cancer. Urology 1998; 52(6): 1085-1089) or B-cell chronic lymphocytic leukemia (Pepper, C., Hoy, T., Bentley, P. Elevated Bcl-2/Bax is a consistent feature of apoptosis resistance in B-cell chronic lymphocytic leukemia and are correlated with vivo chemoresistance. Leuk. Lymphoma 1998; 28(3-4): 355-361). Even reduced expression of Bax alone is associated with poor response rates to radio or chemotherapy in patients with B-cell chronic lymphocytic leukemia (Molica, S., Daftilo, A., Giulino, C., Levato, D., Levato, L. Increased bcl-2/bax ratio in B-cell chronic lymphocytic leukemia is associated with a progressive pattern of disease. Haematologica 1998; 83(12): 1122-1124), breast (Krajewski, S., Blomquist, C., Franssila, K., Krajewska, M., Wasenius, V. M., Niskanen, E., Nordling, S., Reed, J. C. Reduced expression of proapoptotic gene BAX is associated with poor response rates to combination chemotherapy and shorter survival in women with metastatic breast adenocarcinoma. Cancer Res. 1995; 55(19): 4471-4478), ovarian (Tai, Y. T., Lee, S., Niloff, E., Weisman, C., Strobel, T., Cannistra, S. A. BAX protein expression and clinical outcome in epithelial ovarian cancer. J. Clin, Oncol 1998; 16(9): 3211), cervical (Harima, Y., Harima, K., Shikata, N., Oka, A., Ohnishi, T., Tanaka, Y. Bax and Bcl-2 expressions predict response to radiotherapy in human cervical cancer. J. Cancer Res. Clin. Oncology 1998; 124(9): 503-510) and pediatric cancers (McPake, C. R., Tillman, D. M., Poquefte, C. A., George, E. O., Houghton, J. A., Harris, L. C. Bax is an important determinant of chemosensitivity in pediatric tumor cell lines independent of bcl-2 expression and p53 status. Oncol. Res. 1998; 10(5): 235-244). In contrast, increased levels of Bax protein, or increased ratio of Bax/Bcl-2 protein, have been found to be tightly associated with increased therapeutic response (Tai 1998. Harima 1998). Furthermore, it has been suggested that Bax levels also influence the prognosis of human pancreatic cancer: patients whose tumors exhibited Bax immunostaining lived significantly longer (12 months) than those whose tumors were Bax negative (5 months) (Friess, H., Lu, Z., Graber, H. U., Zimmermann, A., Adler, G., Kore, M., Schmid, R. M., Buchler, M. W. Bax but not bcl-2, influences the prognosis of human pancreatic cancer. Gut 1998; 43(3): 414-421). What determines or regulates Bax levels in human cancer cells remains unknown.

Expression of oncogenes that deregulate cell proliferation can also induce apoptosis (White, E. Proc. Soc. Exp. Biol. Med. 1993; 204: 30-39; Harrington, E. A., Fanidi, A., Evan, G. I. Curr. Opin. Genet. Dev. 1994; 4: 120-129), indicating that oncogene expression generates a proapoptotic signal that is present in transformed cells but absent in normal cells. Indeed, most recently, it has been found that an apoptosis-promoting complex consisting caspase-9, Apaf-1 and cytochrome c regulates the process of oncogene-dependent apoptosis (Fearnhead, H. O., Rodriguez, J., Govek, E-. E., Guo, W., Kobayashi, R., Hannon, G., Lazebnik, Y. A. Oncogene-dependent apoptosis is mediated by caspase-9. Proc. Natl. Acad. Sci. 1998; 95: 13664-13669). Since caspase-9, Apaf-1 and cytochrome c are also present in normal cells, it is unclear what is the missing signal in normal cells that triggers activation of the apoptosis-promoting complex.

It would, therefore, be useful to develop a direct method of modulating apoptosis in cells, particularly cancer cells. Further it would be particularly useful to develop a method of inducing apoptosis in cancer cells that acts independently of Bcl-2.

### SUMMARY OF THE INVENTION

According to the present invention, there is provided a calpain enzyme cleaved Bax, amino terminus, 18 kDa fragment for inducing potent cell death activity. Further provided is the method of inducing cell death even in cancer cells overexpressing Bcl-2 oncoprotein. Further provided is a method of using Bax/p18 of triggering cytochrome c release, activation of caspase-3, cleavage of poly(ADP-ribose) polymerase, fragmentation of DNA and induction of cell death. Further provided is a method of introducing Bax/p18 protein into a cancer cell via introducing Bax/p18 cDNA, to cause release of cytochrome c and induction of caspase-3 mediated apoptosis that is not blocked by overexpression of Bcl-2.

### DESCRIPTION OF THE DRAWINGS

Other advantages of the present invention are readily appreciated as the same becomes better understood by reference to the following detailed description when considered in connection with the accompanying drawings wherein:

FIG. 1 shows that Bax cleavage occurred prior to the initiation of the apoptotic execution phase. Jurkat T cells were treated with 50 μM of VP-16 or 1 μM of staurosporin for the indicated hours. At each time point, cells were harvested and used for Western blotting (A-E), DNA fragmentation (F) and TUNEL (G) assays. (A) Caspase-3 processing and activation. Pro-caspase-3 (p32) and an active form of caspase-3 (p17) are indicated. (B) PARP cleavage. The intact PARP (MW 116 kDa) and a PARP cleavage fragment (PARP/p85) are shown. (C) The monoclonal B-9 anti-Bax antibody detects both full-length Bax (Bax/p21) and the cleaved form of Bax (Bax/p18). The p36 band is probably a homeodimer of Bax/p18 (see Discussion). (D) The monoclonal 6A7 anti-Bax antibody detects only the full-length Bax (Bax/p21). Note: the absence of Bax/p18 and p36 bands. (E) Autolysis/activation of the calpain 30-kDa subunit. The calpain 30 kDa subunit (p30) and at least two cleaved, active fragments (˜28 and 22 kDa, respectively) are indicated. (F) DNA fragmentation assay. DNA extracted from cells at each time point was subjected to agarose gel electrophoresis and visualized under UV light. (G) TUNEL assay. The percentages of TUNEL-positive, apoptotic cell population (Ap) are indicated.

FIG. 2 shows cell-free Bax cleavage activity. (2A) This figure shows that Bax/p18 is also present as a cleavage product of Bax/p21. Detection of Bax cleavage activity in protein extracts of cells treated with VP-16 or staurosporin. [³⁵S]methionine-labeled Bax protein was incubated for 2 hours with either buffer alone (as a control, Cl) or a whole cell lysate of Jurkat T cells untreated (0 hours) or treated by VP-16 or staurosporin for the indicated hours, followed by gel electrophoresis and autoradiography. Both labeled full-length (Bax/p21) and the cleaved Bax (Bax/p18) are indicated. (2B) Shows Bax cleavage is a result of calpain activity. Sensitivity of the cell-free Bax cleavage activity to different chemical inhibitors. The ³⁵S-labeled Bax protein was incubated for 2 hours with either buffer alone (Cl) or a whole lysate of Jurkat T cells pretreated with VP-16 for 12 hours, in the absence (−) or presence of a protease inhibitor, including the calpain inhibitor-1 (LLnL, 10 mM), the calpain inhibitor-2 (LLM, 10 mM), the pan-caspase inhibitor Z-VAD-FMK (VAD, 20 mM), the caspase-3 specific inhibitor Ac-DEVD-CMK (DEVD, 20 mM), or the specific proteasome inhibitor b-lactone (Lac, 20 mM). (2C) Shows that Bax cleavage activity is a Ca⁺²-dependent protease activity. Cell-free Bax cleavage assay using a whole cell lysate (W) was performed as in B, in the absence (lane 3, with 10 mM ATP) or presence of 5 mM Ca⁺² (lane 2, with 10 mM ATP), or in the absence of ATP (lane 4, with 5 mM Ca⁺²).

FIG. 3 shows that Bax cleavage occurs in mitochondria. (3A-C) Cytochrome c release. Cytosol (Cyt) and mitochondria (Mit) fractions were prepared from Jurkat T cells treated with either 50 μM of VP-16 or 1 μM of staurosporin for the indicated hours, followed by Western blot assay using a specific antibody to cytochrome c (MW 17 kDa; 3A, 3B). A 40 kDa band, detected in the cytosol fractions and indicated by an arrowhead, was also shown as a loading control (3A). The filter containing the mitochondrial fractions (3B) was reblotted for the mitochondrial cytochrome oxidase subunit II (COX/Mit, MW 26 kDa), which served as a loading control. (3D-F)

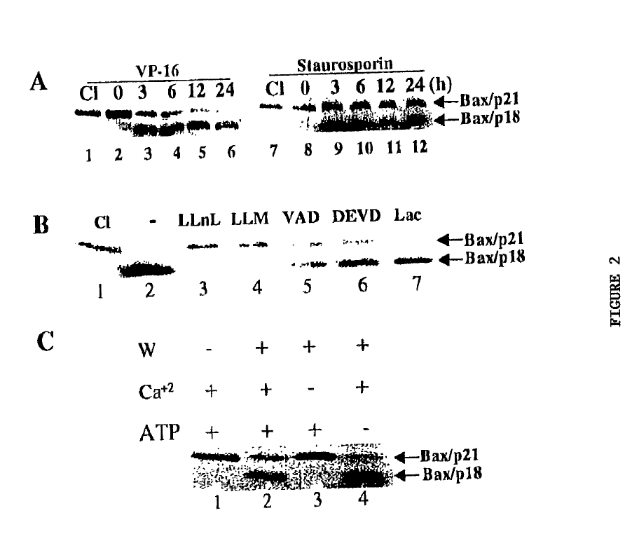

Localization of Bax/p18, Bax/p21 and Bcl-2. Cytosol (Cyt) and mitochondria (Mit) fractions were prepared from Jurkat T cells untreated (0 hours) or treated with VP-16 for 12 or 24 hours, followed by Western blotting with specific antibodies to Bax (the B9 antibody; 3D), Bcl-2 (MW 28 kDa; 3E) or COX (3F). The positions of Bax/p21, Bax/p18, p36, Bcl-2 and COX are indicated. (3G) Bax/p18 does not interact with Bcl-2 in the mitochondrial fraction. The mitochondrial membrane-enriched fraction, prepared from Jurkat T cells either untreated (0 hours) or treated for 12 hours with VP-16, was incubated with a monoclonal anti-Bcl-2 antibody, followed by collecting both immunoprecipitate (IP) and the supernatant (IS) fractions and using them for Western blotting with the B-9 anti-Bax antibody. Both full-length (Bax/p21) and the cleaved (Bax/p18) Bax are indicated. Some IgG chains are shown as a loading control. (3H) The Bax cleavage activity is present in the mitochondrial, but not the cytosol, fraction. [³⁵S]methionine-labeled Bax protein was incubated for 2 hours with either buffer alone (Cl), a whole cell lysate (W), cytosol (Cyt) or mitochondrial (Mit) fraction of Jurkat T cells treated with VP-16 for 12 hours, followed by analysis of Bax cleavage product as described in the legend of FIG. 2.

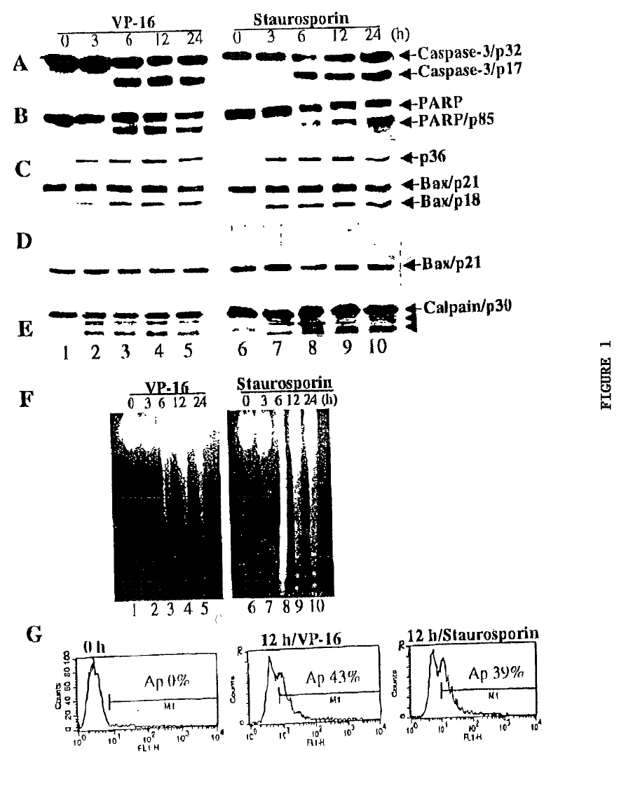

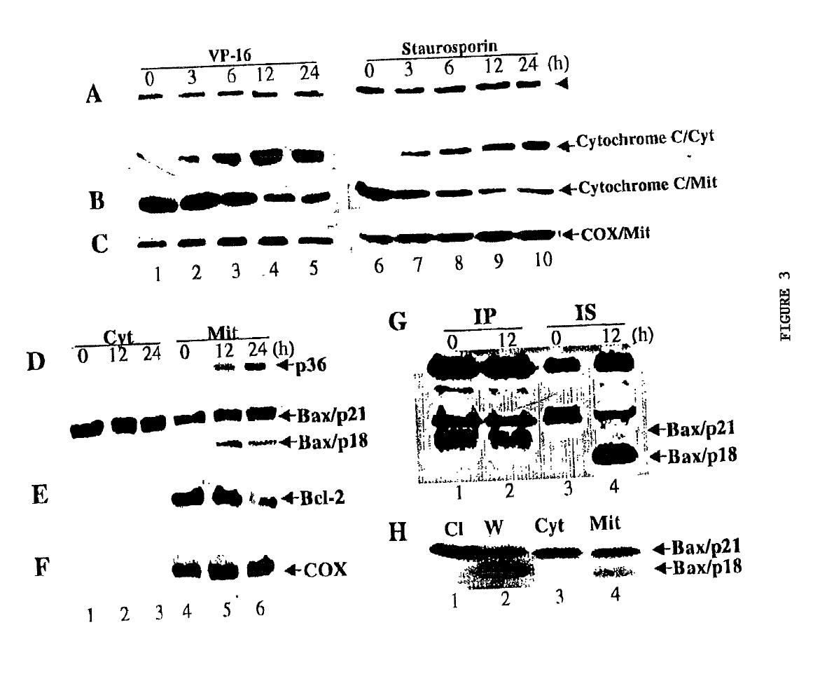

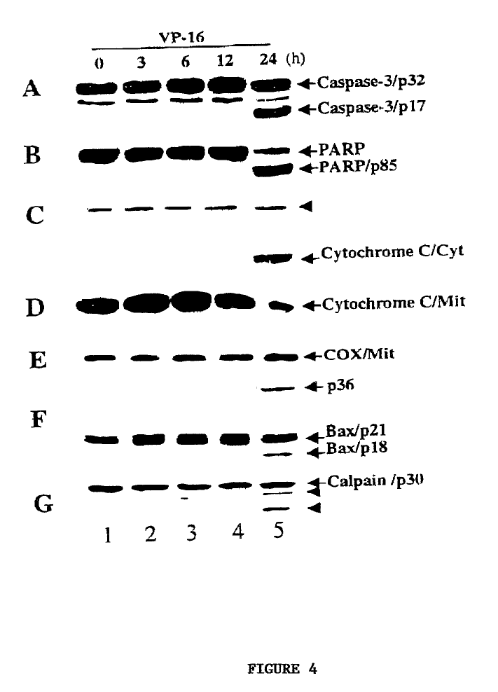

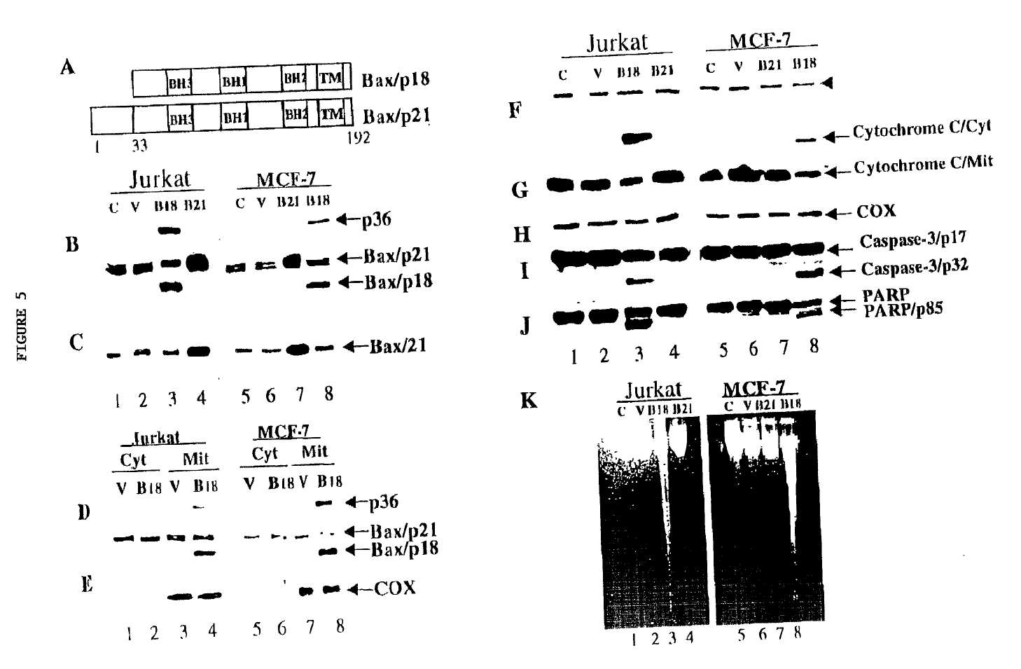

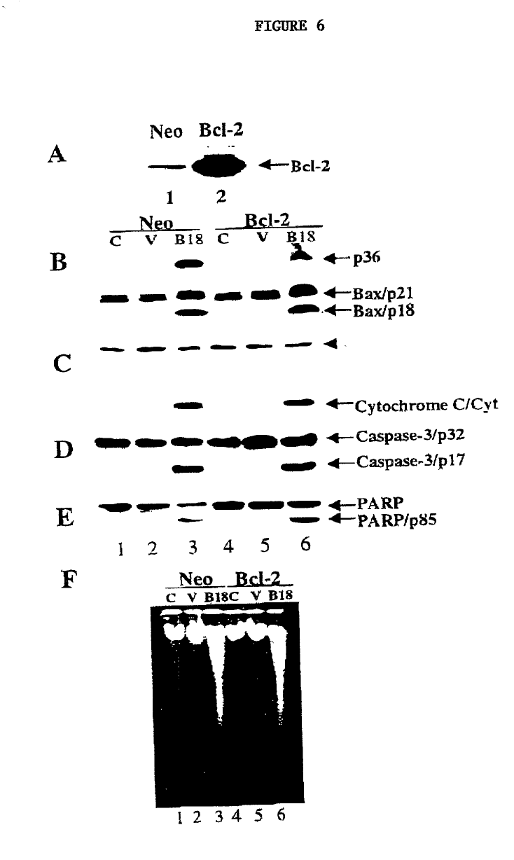

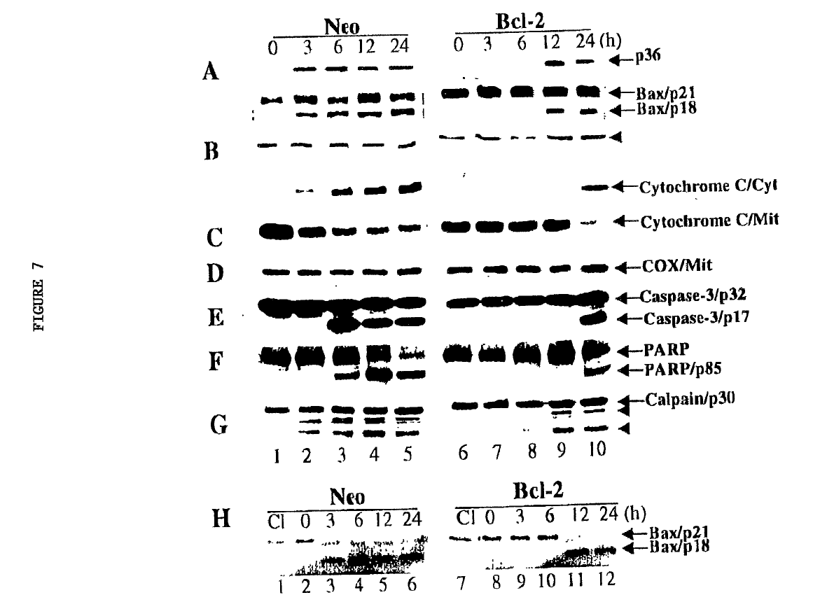

FIG. 4 shows the inhibition of Bax cleavage by the specific calpain inhibitor calpeptin is associated with inhibition of cytochrome c release and apoptosis execution. Jurkat T cells were pre-treated with 10 μM of calpeptin for 1 hour, followed by co-incubation with 50 μM of VP-16 for up to 24 hours, as indicated. At each time point, the cells were collected and used for measurement of caspase-3 processing/activation (4A), PARP cleavage (4B), cytochrome c release (4C-E), Bax cleavage (with B9 antibody; 4F) and calpain 30 kDa subunit autolysis (4G), as described in the legends of FIGS. 1 and 3. FIG. 5 shows overexpression of Bax/p18 in mitochondria induces cytochrome c release and apoptotic cell death. (5A) A schematic diagram of Bax/p18 and full-length Bax protein encoded by the corresponding cDNAs. The cloned Bax/p18 consists of amino acids 33-192 of Bax. The BH1, BH2, BH3 and the transmembrane (TM) domains are indicated. (5B-K) Jurkat T or MCF-7 cells (C for control) were transiently transfected with pcDNA3 vector alone (V) or pcDNA3 containing Bax/p18 (B18) or Bax-α cDNA (B21). After transfection, cells were used for measurement of Bax expression (5B, 5C, with B9 and 6A7 antibodies, respectively) and localization (5D, 5E, with B9 anti-Bax and anti-COX antibodies, respectively), cytochrome c release (5F-G), caspase 3 processing/activation (5I), PARP cleavage (5J) and DNA fragmentation (5K), as described in the legends of FIGS. 1, 3.

FIG. 6 shows that Bax/p18-mediated apoptosis is not blocked by overexpression of Bcl-2. (6A) Bcl-2 levels in exponentially growing Neo and Bcl-2 cells. (6B-F) Both Neo and Bcl-2 cells (6C for control) were transiently transfected with pcDNA3 vector (V) or pcDNA3 vector containing Bax/p18 cDNA (B18), followed by measurement of Bax expression (6B, with B9 antibody), cytochrome c release (6C), caspase 3 processing/activation (6D), PARP cleavage (6E) and DNA fragmentation (6F), as described in FIGS. 1 and 3.

FIG. 7 shows that overexpression of Bcl-2 delays VP-16-induced calpain autolysis, Bax cleavage, cytochrome c release and apoptosis induction. Neo and Bcl-2 cells were treated with 50 μM of VP-16 for indicated hours, followed by measurement of Bax expression (7A, with B9 antibody), cytochrome c release (7B-D), caspase 3 processing/activation (7E), PARP cleavage (7F) and calpain 30 kDa subunit autolysis (7G), as described in FIGS. 1 and 3. An aliquot of the whole cell extract at each time point was also used for cell-free Bax cleavage assay (7H), as described in the legend of FIG. 2.

### DESCRIPTION OF THE INVENTION

Generally, the present invention provides a method of improving cancer treatment. Provided by the current invention is a calpain enzyme cleaved Bax, amino terminus, 18 kDa fragment (Bax/p18) that is a potent proapoptotic molecule that mediates cell death. Further provided is the method of inducing cell death even in cancer cells overexpressing Bcl-2 oncoprotein. Also provided is a method, using Bax/p18, of triggering cytochrome c release, activation of caspase-3, cleavage of poly(ADP-ribose) polymerase, fragmentation of DNA and induction of cell death. Further provided is a method of introducing Bax/p18 protein into a cancer cell via introducing Bax/p18 cDNA, to cause release of cytochrome c and induction of caspase-3 mediated apoptosis that is not blocked by overexpression of Bcl-2.

Applicants have found a novel mechanism by which an active calpain enzyme cleaves Bax at its N-terminus, producing a potent proapoptotic Bax/p18 fragment that in turn induces cytochrome c release and drives programmed cell death. Specifically demonstrated are the following. First, calpain-mediated Bax cleavage occurred in a very early stage of VP-16- or staurosporin-induced apoptosis, which was associated with cytochrome c release but preceded caspase activation and the execution phase of apoptotic cell death (FIGS. 1, 3). Second, the following unique properties of the generated Bax/p18 fragment distinguished itself from the full-length Bax /p21. All the Bax/p18, but only a portion of Bax/p21, were found in the mitochondrial membrane-enriched fraction of drug-treated cells (FIG. 3D). Bax/p18, different from the mitochondrial Bax/p21, did not interact with the antiapoptotic Bcl-2 protein (FIG. 3G). Third, treatment of cells with the specific calpain inhibitor calpeptin inhibited calpain autolysis/activation and Bax cleavage and subsequently delayed the downstream events, including cytochrome c release, caspase activation and apoptotic execution (FIG. 4). Fourth, applicants cloned the Bax/p18 cDNA and transfected it to multiple human cancer cell lines. The overexpressed Bax/p18 protein was found in the mitochondrial fraction, which was accompanied by release of cytochrome c and induction of apoptosis (FIGS. 5, 6), demonstrating that Bax/p18 has the cytochrome c-releasing ability. Fifth, overexpression of Bcl-2 did not block Bax/p18-induced apoptosis (FIG. 6). Finally, Bcl-2 overexpression inhibited drug-induced calpain autolysis/activation, Bax cleavage and cytochrome c-regulated apoptosis (FIG. 7), indicating that at least one of the molecular mechanisms by which Bcl-2 inhibits apoptosis is its ability to inhibit the activation of calpain-mediated Bax cleavage activity.

Early Bax cleavage activity (activity that occurs prior to apoptotic commitment), detected as described herein (FIGS. 1, 3), is a calpain-like activity, which is supported by both in vitro and in vivo evidence: (i) the process of Bax cleavage under cell-free conditions was blocked by addition of calpain inhibitors LLM or LLnL, but not the proteasome inhibitor â-lactone (FIG. 2B); (ii) the cell-free Bax cleavage activity is dependent of Ca⁺² (FIG. 2C); (iii) pretreatment of cells with the specific calpain inhibitor calpeptin delayed the Bax cleavage process (FIG. 4). The delayed, rather than a complete, inhibition by calpeptin (FIG. 4) is due to decreased levels of calpeptin by its metabolism since addition of fresh calpeptin into these cells at 18 hours after VP-16 treatment resulted in a complete inhibition of PARP cleavage at 24 hours.

Early Bax cleavage activity is dependent on calcium (FIG. 2C), inhibitable by specific calpain inhibitors in vitro and in vivo (FIGS. 2B and 4), and associated with autolysis/activation of the 30 kDa subunit of calpain (FIG. 7E). The cloned Bax/p18 cDNA was transfected into several human cancer cell lines. Overexpression of Bax/p18, which was accumulated in the mitochondrial fraction, was sufficient to trigger cytochrome c release and initiated apoptotic execution (FIGS. 3, 5). These data show that the cloned Bax/p18 cDNA encodes a Bax fragment that is identical, or very similar, to the one produced at the early stage of apoptotic process in vivo.

A detailed description of the Bax/p18 fragment activity is set forth in the following non-limiting examples and accompanying Figures, included herewith and incorporated by reference in its entirety.

### EXAMPLES

### MATERIALS AND METHODS

Materials

Etoposide (VP16), staurosporin, proteinase K, Rnase, calpain inhibitor I (LLnL), and calpain inhibitor KK (LLM) were purchased from Sigma Chemical Co. (St. Louis, Mo.). Calpeptin, pan-caspase inhibitor (Z-VAD-FMK) and caspace-3 inhibitor III (Ac-DEVD-CMK) were from Calbiochem (La Jolla, Calif.), and clastolactacystin-lactone was from BIOMOL (Plymouth Meeting, Pa.). L-[³⁵S]methionine was purchased from Amersham (Piscataway, N.J.).

Cell Culture and Treatment

Jurkat T cells transfected with pcDNA vector alone (Neo) or pcDNA vector containing Bcl-2 cDNA (Bcl-2) were gifts from Dr. Hong-gong Wang (Moffitt Cancer Center & Research Institute, Tampa, Fla.). MCF-7, Jurkat T. Neo and Bcl-2 cells were cultured in RPMI 1640 supplemented with 10% fetal calf serum, 100 units/ml of penicillin and 100 μg/ml of streptomycin at 37° C. in a humidified atmosphere consisting of 5% CO₂ and 95% air. These cells were treated with either μM of Staurosporin for the indicated lengths of time in figure legends. For the experiment using a specific calpain inhibitor, Jurkat T cells were pretreated for 1 hour with calpeptin at 10 μM, followed by a co-incubation with 50 μM of VP-16, as indicated in figure legends.

Subcellular Fractionation

Both cytosolic and mitochondria fractions were isolated at 4° C. using a previous protocol (Hockenery et al., 1990) with some modifications. At each time point, cells were washed twice with PBS, resuspended in a hypotonic buffer containing 20 mM HEPES (pH 7.5), 1.5 mM MgC1₂, 5 mM KC1 and 1 mM DTT, and incubated on ice for 10 minutes. The cells were then broken by four passes through a 30 G ½ needle fitted on 1-ml syringe, and the lysate was centrifuged at 2,000×g for 10 minutes. The supernatant was collected and centrifuged again at the same condition. The resulting supernatant was then centrifuged at 14,000×g for 30 minutes, followed by collection of both the supernatant and pellet fractions. The pellet was washed twice with a buffer containing 210 mM mannitol, 70 mM sucrose, 5 mM Tris-HC1 (pH 7.5) and 1 mMEDTA, and resuspended in a lysis buffer (50 mM Tris-HC1, pH 7.5, 5 Mm EDTA, 150 Mm NaCl and 0.5% NP-40) as the mitochondria fraction. The supernatant was further centrifuged at 600,000×g for 30 minutes and the resulting supernatant was collected as the cytosol fraction. To prepare a whole cell extract, cells were lysed in the lysis buffer, and the lysate was centrifuged at 14,000×g for 30 minutes. The supernatant was collected as a whole cell lysate.

Bax/p18 cDNA Cloning and Transfection

Bax/p18 cDNA was amplified by PCR with human Bax-α-cDNA as template using primer 5′-CGTATAAGCTTATGGATCGAGCAGGGCGA (forward) and 5′-CTATCTCGAGTCAGCCCATCTTCTTCCAG (reverse) (Wood et al., 1998) and cloned into pcDNA3.0 between Hind III and Xho I restriction enzyme sites. Bax/p18 cDNA was verified by sequencing. MCF-7, Jurkat T. Neo and BcI-2 cells were transfected with pcDNA3.0 vector alone or pcDNA3.0 vector containing Bax/p18 or Bax/p21 cDNA using GenePORTER Transfection Reagent (Gene Therapy Systems, San Diego, Calif.) according to the manufacture's instructions. Briefly, 3 μg of plasmid DNA in 500 μl of RPMI 1640 medium was incubated for 30 minutes at room temperature with 25 μl of GenePORTER reagent in 500 μl of RPMI 1640 medium. The resultant GenePORTER-plasmid DNA mixture (1 ml) was then added into cells 2×10⁶ cells in a well of a 6-well plate, followed by 5 hours incubation at 37° C. in a humidified atmosphere consisting of 5% CO₂. After that, 1 ml of RPMI 1640 medium containing 20% fetal calf serum was added and the cells were further incubated for additional 43 hours before harvest.

Bax Cleavage Assay

[³⁵S]-labeled bax protein was prepared by using human Bax—pcDNA3 and TNT coupled transcription and translation reticulocyte lysate system (Promega, Madison, Wis.). Bax cleavage assay was performed as described previously (Wood et al., 1998) with some modifications. Briefly, the ³⁵S-labeled Bax protein (1 μl) was incubated with 100 μg of a whole cell extract or a cytosol or mitochondrial fraction in an assay buffer (10 mM HEPES, pH 7.4, 5 mM MgCl₂, 5 mM CaCl₂, and 1 mM DTT), supplemented with ATP-regenerating system (0.1 mg/ml creatine kinase, 100 mM creatine phosphate, and 5 mM ATP) for 2 hours at 37° C. For particular experiments, CaCl₂ or ATP-regenerating system was not added in the cleavage assay system, as described in the figure legends. For inhibitor studies, 10 μM of calpain inhibitor-1 (LLnL), 10 μM of calpain inhibitor-2 (LLM), 20 μM of pan-caspase inhibitor Z-VAD-FMK, 20 μM of caspase-3 inhibitor Ac-DEVD-CMK, or 20 μM of the specific proteasome inhibitor castolactacystin, lactone was added in the Bax cleavage system.

Western Blot Analysis

Equal amounts of protein (30-60 μg) from a whole cell lysate, cytosol or mitochondria fraction were resolved by SDS-polyacrylamide gel electrophoresis and then transferred to the nitrocellulose membranes (Schleicher & Schuell, Keene, N.H.) using a semi-dry transfer system (Bio-Bad, Hercules, Calif.). The membrane was blocked with 5% nonfat dry milk in PBS-Tween (v/v, 0.2%) for 1 hour at room temperature and then incubated with the primary antibody overnight at 4° C. After washing three times with PBS-Tween, the membrane was blotted with the secondary antibody conjugated with horseradish peroxidase for 1 hour at room temperature and washed again. The protein bands were visualized with the enhanced chemiluminescence system (Amersham). The primary antibodies used were: monoclonal B9anti-Bax antibody (Santa Cruz Biotechnology, Santa Cruz, Calif.) at 1:500, monoclonal 6A7 anti-Bax antibody (Pharmingen, San Diego, Calif.) at 1:500, monoclonal anti-Bcl-2 antibody (Dako, Glostrup, Denmark) at 1:500, monoclonal anti-caspase-3 antibody (Onocogene, Cambridge, Mass.) at 1:200, monoclonal anti-cytochrome C antibody (Pharmingen) at 1:500, polyclonal anti-PARP antibody (Boehringer Manheim, Indianapolis, Ind.) at 1:3000, monoclonal anti-cytochrome oxidase unit 11 (COX) antibody (Molecular Probes, Eugene, Oreg.) at 1:200, and monoclonal anti-calpain 30 kDa subunit antibody (Chemicon, Temecula, Calif.) at 1:500. The secondary antibodies used were anti-mouse IgG-horseradish peroxidase and anti-rabbit IgG-horseradish peroxidase (Santa Cruz Biotechnology) at 1:2000.

Immunoprecipitation-Western Blot Assay

Immunoprecipitation assay was performed as described previously (Fattman et al., 1997). Briefly, the mitochondria fraction (500 μg of protein) from VP-16-treated or untreated Jurkat T cells was incubated with a 5 μg of a monoclonal anti-human Bcl-2 antibody overnight at 4° C. The protein-antibody complex was then precipitated by incubating with 2 μl of Protein G plus Protein A agarose (Oncogene) for 1 hour at 4° C. and centrifuging at 2000×g at 4° C. for 1 hour. Both the precipitate and supernatant fractions were then analyzed by immunoblotting using the monoclonal B9 anti-human Bax antibody.

DNA Fragmentation Assay

Cells were re-suspended in a buffer containing 10 mM Tris-HCl (pH 7.4), 10 mM NaCl, 10 Mm EDTA, 1% SDS and 0.5 mg proteinase K. and incubated for 24 hours at 37° C. A DNase-free RNase (Sigma) was then added into the cell lysate at a final concentration of 0.2 mg/ml, followed by an additional incubation at 37° C. for 1 hour. DNA was then precipitated by isopropanol, washed once with 75% ethanol, and dissolved in TE buffer (10 mM Tris-HCl, pH 7.4 and 1 Mm EDTA). Fifteen μg of DNA per sample was subjected to electrophoresis on 1.2% agarose containing 0.5 μg/ml of ethidium bromide.

TdT-Mediated dUTP Nick End Labeling (TUNEL) Assay

TUNEL assay was performed with a Fluorescein-FragEL DNA Fragmentation Detection Kit (Oncogene Research Products), according to manufacturer's instruction. Briefly, cells were fixed in 4% paraformaldehyde for 10 minutes at room temperature and then in 80% ethanol overnight at −20°. After that, cells were washed once with a TBS buffer (20 mM Tris, pH 7.6 and 140 mM NaCl), permeabilized with proteinase K, and labeled with Fluorescein-conjugated dNTP's and TdT enzyme, followed by analysis with flow cytometry.

### RESULTS

Bax is Cleaved Prior to the Apoptotic Execution by a Calpain-Like Activity

RB was internally cleaved by a caspase-like activity in the beginning of the apoptotic execution phase, associated with cleavage of PARP and the internucleosomal fragmentation of DNA (An and Dou, 1996; Fattman et al., 1997; An et al., 1998). However, recently, one group reported that several hours after cleavage of PARP and RB as well as fragmentation of cellular DNA, the pro-apoptotic Bax protein was cleaved into a Bax/p18 fragment by a calpain-like activity (Wood and Newcomb, 1999; Wood et al., 1998). The current inventors measured levels of Bax expression during apoptosis under experimental conditions. Bax cleavage occurred 3 hours prior to the initiation of the apoptotic execution phase (FIG. 1). In this experiment, when human Jurkat T cells were treated with the anticancer agent etoposide/VP-16 (50 μM), the apoptotic execution phase began after 6 hours, as demonstrated by processing/activation of caspase-3 (FIG. 1A, lanes 1-5), cleavage of PARP (FIG. 1B, lanes 15) and RB (An et al., 1998), and fragmentation of cellular DNA measured by both agarose gel electrophoresis (FIG. 1F, lanes 1-5) and TUNEL assay (FIG. 1G).

Levels of Bax protein expression were measured by Western blot analysis of the same cell extracts. A specific monoclonal B-9 antibody that was raised from a Bax fragment of amino acids 1-171 (representing all but the carboxyl terminal 21 amino acids) detected the full-length Bax protein (Bax/p21) in the untreated and treated cells (FIG. 1C). The B9 antibody also detected a band of 18 kDa (Bax/p18) and a band of −36 kDa (p36) in the drug-treated, but not untreated, cells at as early as 3 hours (FIG. 1C, lanes 1-5). The p36 band most likely contains Bax/p18. Another specific monoclonal 6A7 Bax antibody, which reacts with an epitope between amino acids 12-24 of Bax, detected only Bax/p21, but not Bax/p18 nor p36 (FIG. 1D, lanes 1-5), indicating that Bax/p18 is an N-terminal cleaved form of Bax. Therefore, similar to the previous reports (Wood et al., 1998), Bax/p21 was cleaved at its N terminus, resulting in production of a p18 fragment. But in contrast to the previous report (Wood and Newcomb, 1999) in which Bax cleavage occurred several hours after cleavage of PART and RB as well as fragmentation of DNA (referred to as late Bax cleavage), applicants found that Bax was cleaved several hours prior to the initiation of the apoptotic execution (referred to as early Bax cleavage).

To show that Bax/p18 was a cleavage product of Bax/p21, levels of Bax cleavage activity were measured under cell-free conditions using an in vitro-translated, [³⁵S]-labeled Bax protein as substrate (FIG. 2A, lane 1). The Bax cleavage activity was absent in a whole protein extract prepared from exponentially growing Jurkat T cells, but present in preparations from the cells that has been treated with VP-16 for 3 hours or longer (FIG. 2A, lanes 2-6). The kinetics of cell-free Bax cleavage activity matched exactly that of Bax/p18 production in cells treated with VP-16 (compare FIGS. 2A, lanes 2-6, to FIG. 1C, lanes 1-5).

The previously reported late Bax cleavage was mediated by an activated calpain activity (Wood et al., 1998). Because autolysis of the 30 kDa subunit of calpain is associated with its activation during apoptosis (Wood et al., 1998; Nath et al., 1996), applicants measured the kinetics of autolysis/activation of the calpain in the same Jurkat cells treated with VP-16. The autolysis of the calpain 30 kDa subunit, as demonstrated by appearance of two fragments (−28 kDa and −22 kDa), began at 3 hours and continued at later time points (FIG. 1E, lanes 1-5), parallel to the kinetics of Bax cleavage in vivo (FIG. 1C, lanes 1-5) and in vitro (FIG. 2A, lanes 2-6).

To show that Bax cleavage activity was due to calpain, under the experimental conditions, specific inhibitors were used. The cell-free bax cleavage activity in a whole cell extract was completely inhibited by 10 μM of the calpain inhibitor-1 (LLnL) or −2 (LLM), but not by 20 μM of the specific proteasome inhibitor clastolactacystin β-lactone (FIG. 2B, lanes 3, 4, 7 vs. 2), indicating that the early Bax cleavage activity is related to the calpain family. The process of in vitro Bax cleavage was partially inhibited by 20 μM of the general caspase inhibitor Z-VAD-FMK or the relatively specific caspase-3 inhibitor Ac-DEVD-CMK (FIG. 2B, lanes 5, 6 vs. 2), suggesting involvement of caspases in calpain activation. The cell-free Bax cleavage activity was also blocked when Ca⁺² was not added (FIG. 2C, lanes 3 vs. 2), demonstrating that it is a Ca⁺²-dependent protease activity. However, the cell-free Bax cleavage process still occurred when ATP was not added (FIG. 2C, lane 4).

The early Bax cleavage process was also inducible by a non-DNA damage agent. To do so, Jurkat T cells were treated with the kinase inhibitor staurosporin (1 μM) for up to 24 hours. Again, processing/activation of caspase-3, cleavage of PARP, and fragmentation of DNA occurred at 6 hours and later (FIG. 1A, B, F, lanes 6-10, and G). In comparison, Bax cleavage began at 3 hours, which was detected by the monoclonal B9, but not 6A7, anti-Bax antibody (FIG. 1C and D, lanes 6-10). In addition, cell-free bax cleavage activity was also observed when protein extracts were prepared from cells treated with staurosporin for 3 to 24 hours, but not in an untreated cell preparation (FIG. 2A, lanes 7-12). Therefore, under the experimental conditions, prior to the initiation of apoptotic execution, both VP-16 and staurosporin activate a calpain-like enzyme, which in turn cleaves Bax into the Bax/p18 fragment.

Bax Cleavage Occurs in Mitochondria

Applicants investigated the release of mitochondrial cytochrome c into the cytosol induced by the cleavage of bax since the cleavage preceded activation of the effector caspase-3 (FIG. 1C vs. A). Levels of cytochrome c were measured in both cytosolic and membrane-bound (enriched by mitochondria) fractions of the cells that had been treated with either VP-16 or staurosporin for different hours, as described in FIG. 1. No cytosolic cytochrome c was detectable in untreated cells, which appeared after 3 hours of treatment with either VP-16 or staurosporin, and further increased after a longer treatment (FIG. 3A). The increased levels of cytosolic cytochrome c were accompanied by decreased levels of the mitochondrial cytochrome c (FIG. 3B vs. A). The observed cytochrome c release from mitochondria to the cytosol was not an artifact since constitutive levels of a −40 kDa protein band were observed in the cytosolic fractions (indicated by an arrowhead, FIG. 3A; the identity of this protein is unknown) and the mitochondrial cytochrome oxidase (COX; Barrell et al., 1979) in the membrane-bound fractions (FIG. 3C). Importantly, release of cytochrome c was observed 3 hours prior to processing/activation of caspase-3 (FIGS. 3A vs. 1A).

The tight correlation between production of Bax/p18 and release of mitochondrial cytochrome c (FIGS. 1C and 3A) shows that Bax/p18 is a potent pro-apoptotic molecule with a cytochrome c-releasing activity. Therefore, Bax/p18 should be found in the mitochondrial fraction. To test this, both cytosolic and mitochondrial fractions of Jurkat T cells, which were either untreated or treated with VP-16 for 12 or 24 hours, were subjected to Western blot analysis using the specific B9 anti-Bax antibody. In untreated cells, most of Bax/p21 was found in the cytosol fraction; during the drug treatment, the mitochondrial Bax/p21 level was increased (FIG. 3D), as demonstrated previously (Gross et al., 1999). In contrast, both Bax/p18 and p36 proteins were detected only in the mitochondrial fraction of the drug-treated cells (FIG. 3D). Bcl-2 protein was only found in the mitochondria fraction, and the mitochondrial Bcl-2 levels were decreased after a 24 hour treatment with VP-16 (FIG. 3E).

Localization of Bax/p18, Bax/p21 and Bcl-2 in the mitochondrial membrane-enriched fraction (FIG. 3D and E) led to examination of whether they interacted with each other in the mitochondria during the process of VP-16-induced apoptosis. To do so, a coupled Immunoprecipitation-Western blot assay was performed. Mitochondrial fractions were first prepared from Jurkat T cells that were either untreated or treated with VP-16 for 12 hours, followed by preparation of Bcl-2 immunoprecipitates using a specific monoclonal antibody. The obtained Bcl-2 immunoprecipitates (IP) and the immunodepleted supernatant (IS) fractions were then immunoblotted with the B9 anti-Bax antibody (FIG. 3G) All the Bax/p21 protein was co-immunoprecipitated by the anti-Bcl-2 antibody from both untreated and drug-treated cells (FIG. 3G), indicating interaction of Bax/p21 and Bcl-2 in mitochondria under both nonapoptotic and apoptotic conditions. In contrast, all the Bax/p18 fragment was detected only in the anti-Bcl-2-depleted supernatant of the drug-treated cells (FIG. 3G), demonstrating that Bax/p18, although it was also present in mitochondria, did not interact with Bcl-2 protein.

The mitochondrial localization of Bax/p18 could be due to either cleavage of the mitochondrial Bax/p21 by a co-localized calpain activity or cleavage by calpain of the cytosolic Bax/p21, followed by translocation of the resultant Bax/p18 to mitochondria. To distinguish these two possibilities, cell-free Bax cleavage assay was performed using either a mitochondrial, cytosol, or whole cell preparation from VP-16-treated Jurkat T cells. The labeled Bax protein was cleaved by the mitochondrial, but not the cytosolic, fraction (FIG. 3H, lanes 4 vs. 3), indicating the presence of early Bax cleavage activity in the mitochondria of cells treated with VP-16. However, more labeled bax protein was cleaved by the whole cell extract than the mitochondrial fraction (FIG. 3H, lanes 2 vs. 4), suggesting that the mitochondrial Bax cleavage activity could be increased by some cytosolic factor(s) of drug-treated cells. This data suggests that an activated mitochondrial calpain enzyme is probably responsible for cleavage of Bax/p21 into Bax/p18 prior to release of cytochrome c and activation of caspase-3.

Pretreatment with the Specific Calpain Inhibitor Calpeptin Delays VP-16-Induced Calpain Autolysis/Activation, Bax Cleavage, Cytochrome C Release, Caspase-3 Processing/Activation and Apoptosis.

Activation of the calpain-like activity and subsequent cleavage of bax preceding cytochrome c-associated apoptosis induction (FIGS. 1, 3) suggested a critical role of Bax/p18 in committing a cell to releasing cytochrome c and undergoing apoptosis. If so, a specific calpain inhibitor should be able to prevent Bax from being cleaved and consequently inhibit the mitochondrial cytochrome c release and apoptosis. This was tested by pretreating Jurkat T cells for 1 hour with 10 μM of a specific calpain inhibitor calpeptin (Medhi, 1991), which itself did not induce apoptosis (see FIG. 4, lane1; Wood et al., 1998). After that, 50 μM of VP-16 was added (in the presence of calpeptin) and the cells were then incubated for up to 24 hours. The pre- and co-treatment with calpeptin delayed the VP-16-induced caspase-3 processing and PARP cleavage from 6 to 24 hours (FIG. 4 vs. 1, A and B, lanes 1-5). The delayed caspase-3 activation was likely due to a delay in release of cytochrome c from mitochondria to the cytosol (FIG. 4, C-E, vs. FIG. 3, A-C, lanes 1-5). In addition, the delayed cytochrome c release was also associated with a delay in the process of Bax cleavage (FIGS. 4F vs. 1C, lanes 1-5). Consistent with the delay in Bax cleavage, autolysis of the 30 kDa small subunit of calpain was also delayed from 3 hours to 24 hours (FIGS. 4G vs. 1E, lanes 1-5). These data suggest that autolysis of calpain is required for its activation and that the activated calpain is required for Bax cleavage. Taken together, the data suggest that an active mitochondrial calpain enzyme is likely responsible for the early Bax cleavage and that the produced Bax/p18 mediates the cytochrome c-regulated apoptotic process.

Overexpression of Bax/p18 in Mitochondria is Sufficient to Induce Cytochrome C Release and Apoptotic Cell Death

Early Bax cleavage activity was similar to the late Bax cleavage activity reported previously (Wood et al., 1998). The Bax cleavage site for the late calpain activity was identified to be around amino acids 30-33 (FIQD) of Bax (Wood et al., 1998). PCR techniques were used to clone the cDNA sequences corresponding to a fragment of amino acids 33-192 of Bax (FIG. 5A). The Bax/p18 cDNA was then subcloned into a pcDNA3.0 vector.

To directly test the idea that the cloned Bax/p18 is a mediator of cytochrome c release and apoptosis, Jurkat T cells (control in FIG. 5B, lane 1) were transiently transfected with either pcDNA3.0 vector alone (V) or pcDNA3.0 vector containing Bax/p18 (B18) or Bax/p21 cDNA (B21). After 48 hours transfection, cells were harvested for measurement of Bax expression, Bax localization, cytochrome c release and apoptosis (FIG. 5). Western blotting using the monoclonal B9 anti-Bax antibody revealed high levels of Bax/p18 and p36 proteins in the Bax/p18 cDNA-transfected cells (FIG. 5B, lane 3), demonstrating that p36 contains Bax/p18. High levels of Bax/p21 protein were observed in the full-length Bax cDNA-transfected cells (FIG. 5B, lane 4). Overexpression of Bax/p21, but not Bax/p18, was also detected by the monoclonal 6A7 Bax antibody (FIG. 5C, lanes 1-4), consistent with that the transfected Bax/p18 did not contain the N-terminal sequence.

To examine the localization of the transfected Bax/p18 protein, both cytosolic and mitochondrial fractions were prepared from Jurkat T cells transiently transfected with either vector alone or Bax/p18 cDNA, and analyzed by Western blotting using the B9 Bax antibody. All the transfected Bax/p18 protein was found in the mitochondrial, but not the cytosolic, fraction (FIG. 5D, lanes 4 vs. 2). The p36 band was also found only in the mitochondrial fraction of the Bax/p18 CDNA transfected cells (FIG. 5D, lanes 4 vs. 2).

To directly test whether Bax/p18 has the cytochrome c-releasing ability, levels of both cytosolic and mitochondrial cytochrome c were measured in the transfected Jurkat T cells. Release of cytochrome c was induced in the cells transfected with Bax/p18 cDNA, as demonstrated by an increased level of the cytosolic cytochrome c and a decreased level of the mitochondrial cytochrome c (FIG. 5, F-H, lanes 1-3). In contrast, cytochrome c release was not observed in the cells transfected with the Bax/p21 cDNA (FIGS. 5, F and G, lanes 4 vs. 2). Furthermore, caspase-3 processing/activation, PARP cleavage and DNA fragmentation (FIGS. 51 and J, lanes 1-4) were also detected only in the cells transfected with Bax/p18, but not Bax/p21, cDNA.

To confirm the cytochrome c-releasing and apoptosis-inducing ability of Bax/p18, another cell line, human breast cancer MCF-7, was transiently transfected with Bax/p18 cDNA, using the vector (V) and Bax/p21 cDNA plasmid as controls (FIGS. 5, B-J, lanes 5-8). Again, high levels of Bax/p18 and p36 were found in the Bax/p18 cDNA-transfected MCF-7 cells, and high levels of full-length Bax protein were in the Bax cDNA-transfected MCF-7 cells (FIGS. 5B and C, lanes 5-8). All the overexpressed Bax/p18 and p36 in MCF-7 cells were again found in the mitochondrial preparation (FIGS. 5D and E, lanes 5-8). The mitochondrial localization of Bax/p18 in MCF-7 cells were associated with release of cytochrome c, cleavage of PARP and fragmentation of DNA (FIG. 5, F-J, lanes 5-8). Overexpression of Bax/p21 in MCF-7 cells again did not induce cytochrome c-associated apoptotic events (FIG. 5, F-J, lanes 7 vs. 6). Therefore, transfection of Bax/p18 cDNA in both Jurkat and MCF-7 cells targeted Bax/p18 protein to mitochondria, which was sufficient to induce cytochrome c release and subsequent apoptotic cell death. From these data, it is concluded that Bax/p18 has the cytochrome c-releasing ability.

Bax/p18-Induced Apoptosis Cannot be Blocked by Overexpression of Bcl-2

The lack of interaction between Bax/p18 and Bcl-2 proteins in the mitochondrial fraction under apoptotic conditions (FIG. 5G) shows that Bax/p18-induced cytochrome c release and apoptotic cell death was independent of Bcl-2. Therefore, Bcl-2- and the vector-overexpressing cells should be equally sensitive to apoptosis induction by Bax/p18 transfection. To demonstrate this, Jurkat T cells overexpressing Bcl-2 or vector alone (Neo) (FIG. 5A) were transiently transfected with pcDNA vector (V) or pcDNA vector containing Bax/p18 cDNA (B18), followed by measurement of Bax expression, Bax localization, cytochrome c release and apoptosis induction. High levels of Bax/p18 and p36 proteins were found in Bcl-2 overexpressing cells transfected with the Bax/p18 cDNA, which were comparable to that in the Bax/p18-transfected Neo cells (FIG. 5B, lanes 6 vs. 3). Similar high levels of cytosolic cytochrome c were observed in both Bcl-2 and Neo cell lines transfected with the Bax/p18 cDNA, but not the pcDNA vector (FIG. 5C, lanes 1-6). Furthermore, similar levels of induced apoptosis were also obtained in both Bcl-2 and Neo cell lines transfected with the Bax/p18 cDNA but not the pcDNA vector, as demonstrated by processing of caspase-3, cleavage of PARP, and fragmentation of DNA (FIG. 5, D-F, lanes 1-6). These data demonstrate that Bcl-2 is unable to block Bax/p18-mediated cytochrome c release and apoptosis.

Overexpression of Bcl-2 Protein Delays VP-16-Induced Calpain Autolysis, Bax Cleavage, Cytochrome C Release and Apoptosis Induction

Although Bcl-2 overexpression did not block Bax/p18-mediated apoptosis (FIG. 72), Bcl-2 had been shown to repress the cellular apoptosis process triggered by a diverse array of stimuli including chemotherapeutic anti-cancer drugs (Green and Reed, 1998; Adams and Cory, 1998; Gross et al., 1999). If production of Bax/p18 was important for apoptosis induced by VP-16 (FIG. 1), the drug-induced Bax cleavage process should be inhibited in cells overexpressing Bcl-2. To demonstrate this, both Bcl-2 and Neo cells were treated with 50 μM of VP-16 for 3, 6, 12 or 24 hours. At each time point, aliquots of the cells were used to assay expression of Bax, release of cytochrome c, and induction of apoptosis. Kinetics of apoptosis-associated events induced in Neo cells was virtually identical to that in parental Jurkat cells (FIGS. 7 vs. 1, 3). In both Neo and parental cell lines treated with VP-16, appearance of Bax/p18 and p36 bands were detected at as early as 3 hours (FIGS. 7A vs. 1C, lanes 1-5), associated with increased levels of cytosolic cytochrome c and decreased levels of mitochondrial cytochrome c (FIGS. 7B-D vs. 3A-C, lanes 1-5). In addition, after 6 hours or longer treatment of both Neo and Jurkat cell lines, apoptosis was induced, as evident by processing of caspase-3 and cleavage of PARP (FIG. 7E and F vs. FIG. 7A and B, lanes 1-5). However, all these VP-16-induced events were delayed in Bcl-2 overexpressing cells. Appearance of Bax/p18 and p36 bands were observed in Bcl-2 cells at 12 and 24 hours of VP-16 treatment (FIGS. 7A, lanes 6-10), indicating a 9 hour delay (FIG. 7A, lanes 9 vs. 2). Consistent with that, release of cytochrome c was detected after 12 hours in Bcl-2 cells treated with VP-16 (FIGS. 7B-D, lanes 6-10). Associated with the delayed cytochrome c release, both caspase-3 processing and PARP cleavage were observed only after 24 hours VP-16 treatment of Bcl-2 cells (FIG. 7E and F, lanes 6-10). These data show that Bcl-2 overexpression delays VP-16-induced cleavage of Bax/p21 into Bax/p18, and subsequently inhibits cytochrome c-dependent apoptosis.

To investigate whether the delayed events observed in Bcl-2 overexpressing cells were due to inhibition of calpain activation, levels of calpain autoproteolysis and cell-free Bax cleavage activity were measured in the Bcl-2 and Neo cells that had been treated with VP-16 for different hours. Overexpression of Bcl-2 delayed the autolysis process of calpain 30 kDa subunit from 3 to 12 hours (FIG. 7G, lanes 9 vs. 2). In addition, detection of cell-free Bax cleavage activity was also delayed by 9 hours in Bcl-2 overexpressing vs. Neo cells (FIG. 7H, lanes 11 vs. 3). The delayed calpain autolysis and Bax cleavage activity in Bcl-2 vs. Neo cells were paralleled to the delayed Bax cleavage process measured in vivo (FIG. 7, G and H vs. A). Therefore, Bcl-2 overexpression delayed VP-16-induced calpain activation and subsequently Bax cleavage. Since the levels of overexpressed Bcl-2 protein remained unchanged during the VP-16 treatment, it is possible that activation of Bax cleavage enzyme calpain, although inhibitable by Bcl-2 overexpression, can also be induced by VP-16 via a Bcl-2-independent pathway. Taken together, the data shows that Bax/p18, generated by an active calpain enzyme, promotes cytochrome c release and induces cell death.

### DISCUSSION

It has been suggested that apoptosis associated events occurring at the level of Bax protein include translocation to mitochondria (Hus et al., 1997; Wolter et al., 1997), change in conformation (Desagher et al., 1999), oligomerization (Goping et al., 1998; Gross et al., 1998), membrane integration (Goping et al., 1998; Gross et al., 1998), and cooperation in some of the above processes with other proteins such as Bid (Eskes et al., 2000; Desagher et al., 1999). Although the molecular mechanisms by which Bax stimulates cytochrome c efflux are still unknown.

Applicants demonstrate a novel mechanism by which an active calpain enzyme cleaves Bax at its N-terminus, producing a potent proapoptotic Bax/p18 fragment that in turn induces cytochrome c release and drives programmed cell death. Specifically, there is reported the following new findings.

First, calpain-mediated Bax cleavage occurred in a very early stage of VP-16 or staurosporin-induced apoptosis, which was associated with cytochrome c release but preceded caspase activation and the execution phase of apoptotic cell death (FIGS. 1, 3).

Second, the following unique properties of the generated Bax/p18 fragment distinguished itself from the full-length Bax/p21. All the Bax/p18, but only a portion of Bax/p21, were found in the mitochondrial membrane-enriched fraction of drug-treated cells (FIG. 3D). Bax/p18, different from the mitochondrial Bax/p21, did not interact with the antiapoptotic Bcl-2 protein (FIG. 3G).

Third, treatment of cells with the specific calpain inhibitor calpeptin inhibited calpain autolysis/activation and Bax cleavage, and subsequently delayed the downstream events, including cytochrome c release, caspase activation and apoptotic execution (FIG. 4).

Fourth, the Bax/p18 cDNA was closed and transfected it to multiple human cancer cell lines. The overexpressed Bax/p18 protein was found in the mitochondrial fraction, which was accompanied by release of cytochrome c and induction of apoptosis (FIGS. 5, 6), demonstrating that Bax/p18 has the cytochrome c-releasing ability.

Fifth, overexpression of Bcl-2 did not block Bax/p18-induced apoptosis (FIG. 7).

Finally, Bcl-2 overexpression inhibited drug-induced calpain autolysis/activation, Bax cleavage and cytochrome c-regulated apoptosis (FIG. 7), indicating that at least one of the molecular mechanisms by which Bcl-2 inhibits apoptosis is its ability to inhibit the activation of calpain-mediated Bax cleavage activity.

Inhibition of drug-induced Bax cleavage by either calpeptin pretreatment or Bcl-2 overexpression resulted in inhibition of cytochrome c release and apoptotic cell death (FIGS. 4, 7). In addition, overexpression of Bax/p18 cDNA in multiple cancer cell lines was sufficient to trigger release of cytochrome c and induce apoptosis, which was not prevented by overexpression of Bcl-2 protein (FIGS. 5, 6). Furthermore, Bax/p18, produced by either drug treatment or transfection, was found in the mitochrondrial membrane-enriched, but not the cytosol, fraction (FIGS. 3, 5). Unlike the full-length Bax, the Bax/p18 did not interact with Bcl-2 protein in the mitochondrial fraction (FIG. 3G). The data are consistent with an early report that deletion of the N-terminal 20 amino acids of Bax enabled its targeting to mitochondria in vitro (Goping et al., 1998), although such a Bax mutation has never been reported in vivo. In contrast to that, Bax/p18 can be generated in a cell after apoptotic death program was triggered (FIGS. 1, 7).

The results do not rule out the possibility that dimerization or a post-translational modification of Bax/p18 is necessary for its proapoptotic function. Kinetically, whenever Bax/p18 was generated (i.e., by either VP-16 or staurosporin treatment), a p36 band was observed (FIGS. 1, 7), whereas whenever production of Bax/-18 was inhibited or delayed (i.e., by calpeptin treatment or Bcl-2 overexpression), appearance of p36 was undetected or delayed (FIGS. 4, 7). In addition, both Bax/p18 and p36 proteins were found in the mitochondrial membrane-enriched, but not the cytosol, fraction of drug-treated Jurkat cells (FIG. 3). Most importantly, transfection of different cell lines with Bax/p18, but not Bax/-21, cDNA resulted in generation of p36 band (FIGS. 5, 6), and both overexpressed Bax/p18 and p36 proteins were found in the mitochondrial, but not cytosol, fraction of these cells (FIG. 5D). Only under certain in vitro conditions, detection of Bax/p18 was uncoupled from that of p36. For example, in the anti-Bcl-2-depleted supernatant fraction, while an abundant band of Bax/p18 was observed, p27 was not apparent (FIG. 3G), suggesting dissociation of p36 into Bax/p18 during the Immunoprecipitation process. In addition, when a labeled Bax/p18 was generated by in vitro incubation with a protein extract of cells treated with VP-16 or staurosporin, no labeled p36 was detected.

The early Bax cleavage activity, detected under the experimental conditions (FIGS. 2, 7), is a calpain-like activity, which is supported by both in vitro and in vivo evidence: (i) the process of Bax cleavage under cell-free conditions was blocked by addition of calpain inhibitors LLM or LLnL, but not the proteasome inhibitor â-lactone (FIG. 2B); (ii) the cell-free Bax cleavage activity is dependent of Ca⁺² (FIG. 2C); (iii) pretreatment of cells with the specific calpain inhibitor calpeptin delayed the Bax cleavage process (FIG. 4). The delayed, rather than a complete, inhibition by calpeptin (FIG. 4) is probably due to decreased levels of calpeptin by its metabolism (FIG. 4G, lane 11) since addition of fresh calpeptin into these cells at 18 hours after VP-16 treatment resulted in a complete inhibition of PARP cleavage at 24 hours.

The Bax/p18 cDNA was cloned based on the reported Bax cleavage site (Wood and Newcomb, 1999), and transfected it to several human cancer cell lines. Indeed, overexpression of Bax/p18, which was accumulated in the mitochondrial fraction, was sufficient to trigger cytochrome c release and initiated apoptotic execution (FIGS. 5, 6). These data show that the cloned Bax/p18 cDNA encodes a Bax fragment that is similar, to the one produced at the early stage of apoptotic process (FIG. 1C).

Under cell-free conditions, the Bax cleavage activity was absent in a protein extract of growing tumor cells, but present in a preparation of cells treated with an apoptotic stimulus (FIGS. 2A, 13H). In addition, the Bax cleavage activity appears to be located in mitochondria because the mitochondrial membrane-enriched, but not the cytosol, fraction was able to cleave a labeled Bax/p21 into Bax/p18 (FIG. 2C). However, some cytosolic factors can be required for up-regulation of Bax cleavage activity since a whole cell extract cleaved more labeled Bax protein than a mitochondrial fraction (FIG. 2C). These cytosolic factors might regulate either conformation of Bax or levels of calpain and/or calpain inhibitors. Addition of a caspase inhibitor partially inhibited the cell-free Bax cleavage activity (FIG. 2B), suggesting that caspases positively regulate the process of Bax cleavage. In addition, overexpression of Bcl-2 delayed activation of calpain, the Bax cleavage enzyme (FIGS. 7G and H), indicating that Bcl-2 negatively regulates the Bax cleavage process. The delayed, rather than a complete, inhibition by Bcl-2 (FIG. 7), together with the observation that levels of overexpressed Bcl-2 protein were unchanged during the VP-16 treatment (data not shown), suggests existence of another pathway(s) for activation of Bax cleavage enzyme that is VP-16-inducable but Bcl-2-independent.

Finally, and most importantly, different results were obtained from that previously reported using the specific calpain inhibitor calpeptin. Calpeptin treatment delayed drug-induced calpain autolysis, Bax cleavage, cytochrome c release, caspase-3 activation and PARP cleavage (FIG. 4). In contrast, Wood and Newcomb reported that calpeptin inhibited only drug-induced calpain autolysis and Bax cleavage, but not PARP cleavage and DNA fragmentation (Wood and Newcomb, 1999). Therefore, the early Bax cleavage plays a causative role in apoptosis, whereas the late Bax cleavage is a result of caspase activation (Wood and Newcomb, 1999).

Transfection of Bax/p21 cDNA did not induce apoptosis in either Jurkat T or MCF-7 cells (FIG. 5), which was different from some previous reports (Pastorino et al., 1998; Pastorino et al., 1999). The full-length Bax/p21 was also reported to be localized mainly in the cytosol under non-apoptotic conditions (Gross et al., 1999). The inventors found that the endogenous Bax/p21 was associated with Bcl-2 in the mitochondrial fraction (FIG. 3G). The data shows that the Bax/p18 fragment is a more potent apoptosis inducer than the full-length Bax protein.

Delivery of Therapeutics (Compound)

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred.

Throughout this application, various publications, including United States patents; are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1. A composition for inducing tumor cell death comprising an amino terminus 18 kDa Bax protein fragment.

2. A composition for inducing tumor cell death comprising a Bax/p18 cDNA.

3. A method for inducing tumor cell death comprising administering an effective amount of the composition of claim 1.

4. A method for inducing tumor cell death comprising administering an effective amount of the composition of claim 2.

5. A method for triggering cytochrome c mediated cell death comprising administering an effective amount of the composition of claim 1.

6. A method for triggering cytochrome c mediated cell death comprising administering an effective amount of the composition of claim 2.

7. A method of overcoming Bcl-2 apoptosis resistance comprising administering an effective amount of the composition of claim 1.

8. A method of overcoming Bcl-2 apoptosis resistance comprising administering an effective amount of the composition of claim 2.

9. A method of inducing cancer cell death by administering a compound with calpain activity in an amount effective to cleave Bax to produce a Bax/p18 protein fragment.
